SEQUENCE LISTING (24 sequences)

SEQ ID NO: 1 - DNA, 25 bp, Artificial
  forward primer for Oct4
gacaacaatgaaaatcttcaggaga

SEQ ID NO: 2 - DNA, 23 bp, Artificial
  reverse primer for Oct4
ttctggcgccggttacagaacca

SEQ ID NO: 3 - DNA, 24 bp, Artificial
  forward primer for Alb
tgcttgaatgtgctgatgacaggg

SEQ ID NO: 4 - DNA, 25 bp, Artificial
  reverse primer for Alb
aaggcaagtcagcaggcatctcatc

SEQ ID NO: 5 - DNA, 24 bp, Artificial
  forward primer for AFP
tgcagccaaagtgaagagggaaga

SEQ ID NO: 6 - DNA, 24 bp, Artificial
  reverse primer for AFP
catagcgagcagcccaaagaagaa

SEQ ID NO: 7 - DNA, 20 bp, Artificial
  forward primer for CK-19
atggccgagcagaaccggaa

SEQ ID NO: 8 - DNA, 20 bp, Artificial
  reverse primer for CK-19
ccatgagccgctggtactcc

SEQ ID NO: 9 - DNA, 17 bp, Artificial
  forward primer for Vim
cacctacagcctctacg

SEQ ID NO: 10 - DNA, 16 bp, Artificial
  reverse primer for Vim
agcggtcattcagctc

SEQ ID NO: 11 - DNA, 19 bp, Artificial
  forward primer alpha-SMA
agtacccgatagaacatgg

SEQ ID NO: 12 - DNA, 16 bp, Artificial
  reverse primer alpha-SMA
ttttctcccggttggc

SEQ ID NO: 13 - DNA, 20 bp, Artificial
  foward primer for CYP1B1
caccaaggctgagacagtga

SEQ ID NO: 14 - DNA, 20 bp, Artificial
  reverse primer for CYP1B1
gccaggtaaactccaagcac

SEQ ID NO: 15 - DNA, 20 bp, Artificial
  forward primer for CYP2C9
ggacagagacgacaagcaca

SEQ ID NO: 16 - DNA, 19 bp, Artificial
  reverse primer for CYP2C9
tggtggggagaaggtcaat

SEQ ID NO: 17 - DNA, 20 bp, Artificial
  Forward primer for CYP3A4
tgtgcctgagaacaccagag

SEQ ID NO: 18 - DNA, 20 bp, Artificial
  reverse primer for CYP3A4
gcagaggagccaaatctacc

SEQ ID NO: 19 - DNA, 20 bp, Artificial
  forward primer for CYP2E1
ccgcaagcattttgactaca

SEQ ID NO: 20 - DNA, 20 bp, Artificial
  reverse primer for CYP2E1
gctccttcaccctttcagac

SEQ ID NO: 21 - DNA, 20 bp, Artificial
  foward primer for CYP1A1
aggcttttacatccccaagg

SEQ ID NO: 22 - DNA, 20 bp, Artificial
  reverse primer for CYP1A1
gcaatggtctcaccgataca

SEQ ID NO: 23 - DNA, 19 bp, Artificial
  foward primer for beta-Actin
tcaccaccacggccgagcg

SEQ ID NO: 24 - DNA, 20 bp, Artificial
  reverse primer for beta-Actin
tctccttctgcatcctgtcg

(19) United States
(12) Patent Application Publication
(10) Pub. No.: US 2012/0189702 A1
(43) Pub. Date: Jul. 26, 2012

(54) CELL THERAPY FOR TREATMENT OF LIVER FAILURE

(75) Inventor: Sanjeev Gupta, Scarsdale, NY (US)

(73) Assignee: Albert Einstein College of Medicine of Yeshiva University, Bronx, NY (US)

(21) Appl. No.: 13/387,796

(22) PCT Filed: Jul. 16, 2010
(86) PCT No.: PCT/US10/02009

§ 371 Date: Apr. 9, 2012

Related U.S. Application Data
(60) Provisional application No. 61/273,758, filed on Aug. 6, 2009.

(51) Int. Cl. A61K 35/407; A61K 9/14; A61P 1/16; A61K 35/12

(52) U.S. Cl. 424/491; 424/93.7; 424/490

(57) ABSTRACT

Disclosed are methods for treating liver failure in a subject comprising transplanting hepatocytes or stem or progenitor cells in an extrahepatic site in the subject in an amount sufficient to provide liver support and/or induce liver regeneration, where the transplanted hepatocytes or stem or progenitor cells are used along with extracellular matrix-coated microcarriers.

### CROSS-REFERENCE TO RELATED APPLICATION

This application claims the benefit of U.S. Provisional Patent Application No. 61/273,758, filed Aug. 6, 2009, the content of which is hereby incorporated by reference into the subject application.

### STATEMENT OF GOVERNMENT SUPPORT

This invention was made with government support under grant numbers R01 DK46952 and R01 DK071111 awarded by the National Institutes of Health. The government has certain rights in the invention.

### BACKGROUND OF THE INVENTION

Throughout this application various publications are referred to in parenthesis. Full citations for these references may be found at the end of the specification immediately preceding the claims. The disclosures of these publications are hereby incorporated by reference in their entireties into the subject application to more fully describe the art to which the subject application pertains.

Liver-directed cell therapy constitutes an important paradigm for genetic and acquired conditions. Correction of many genetic disorders requires significant repopulation of the organ with transplanted cells, which may be accomplished over time (Wu et al., 2008). In other states, e.g., acute liver failure, where mortalities are high and the need for therapy is immediate, replacement of the whole liver is not always possible, for example because donor organs are in short supply and liver transplantation may be prevented by irreversible complications, technical complexities, or unavailability of transplantation programs (Murray et al., 2008). Since suitable cells may be banked and more easily transplanted, cell therapy represents an attractive alternative (Fisher and Strom, 2006). However, the molecular pathophysiology of acute liver failure is incompletely understood, partly because liver injury arises from multiple and varied causes. It has been unknown whether reseeding of the liver with transplanted cells is critical or whether extrahepatic support from transplanted cells will suffice for liver regeneration. This distinction is important because reseeding of the liver requires deposition of cells in liver sinusoids, which have limited capacity, and transplanted cells need several days to integrate and longer to proliferate in the liver parenchyma (Gupta et al., 2000; Rajvanshi et al., 1996). Also, cell transplantation in liver sinusoids produces hepatic injury and inflammation, which may worsen liver failure (Gupta et al., 2000; Joseph et al., 2002; Krohn et al., 2009). However, cells may be transplanted in extrahepatic sites, e.g., peritoneal cavity, where cells retain suitable functions, including secretion of proteins in blood (Demetriou et al., 1986; Gupta et al., 1994; Kumaran et al., 2005). The present application address the need of cell therapy for liver failure.

### SUMMARY OF THE INVENTION

The present invention provides methods for treating liver failure in a subject comprising transplanting hepatocytes or stem cells or progenitor cells in an extrahepatic site in the subject in an amount sufficient to induce liver regeneration, wherein the transplanted hepatocytes or stem cells or progenitor cells are attached to extracellular matrix-coated microcarriers. Additional objects of the invention will be apparent from the description that follows.

### BRIEF DESCRIPTION OF THE DRAWINGS

FIG. 1A-1D. Changes in parameters of liver injury. Shown are vehicle-treated control NOD.CB17-Prkdc^{scid}/J mice, and NOD.CB17-Prkdc^{scid}/J mice 1 d and 6 d after either 125 or 150 mg/kg monocrotaline (MCT) following priming with rifampicin (Rif) and phenyloin (Phen) for 3 d. (A) Shows changes in serum ALT with higher ALT levels in drug-treated mice. (B) Changes in serum bilirubin levels. (C) Showing extent of encephalopathy in mice. (D) Showing survival curves with extensive mortality in drug-treated mice.

FIG. 2A-2D. Hepatic damage in NOD.CB17-Prkdc^{scid}/J mice. (A) Shows normal liver from vehicle-treated control mouse. (B) Shows loss of liver parenchyma (areas without nuclei) 1 d after Rif, Phen and 125 mg/kg MCT. (C) Liver 3 d after drugs with some recovery. (D) Shows severe loss of liver parenchyma 7 d after drugs. Orig. Mag., ×100, Hematoxylin &Eosin (H&E) stain.

FIG. 3A-3C. Cell therapy outcomes in acute liver failure. Shown are NOD.CB17-Prkdc^{scid}/J mice treated with microcarriers alone (sham-treated) and intraperitoneal transplantation of microcarriers and Fischer 344 (F344) rat hepatocytes. (a) Survival curves showing mortality, which was reversed after transplantation of 5, 10 or 50×10⁶ hepatocytes. (b) Shows encephalopathy in sham-treated mice and its absence in mice treated with cell transplantation. (c) Prothrombin time was prolonged throughout in sham-treated mice, whereas 5-10 d after cell transplantation, prothrombin time was lowered, although not to normal levels in mice treated with cells.

FIG. 4A-4J. Fate of transplanted and native liver cells after cell therapy in mice with liver failure. (a) Shows healthy transplanted F344 rat hepatocytes and microcarriers (mc) in cell-microcarrier conglomerates recovered after 14 d from peritoneal cavity. (b) Histochemical staining showing glycogen in transplanted hepatocytes in peritoneal cavity. (c-f) Beta-galactosidase (LacZ) staining (blue color) in C57BL/6J-^{Gtrosa26tm1Sor} (Rosa26) donor mouse liver containing Eschericia coli β-galactosidase gene (c), its absence in recipient NOD.CB17-Prkdc^{scid}/J mouse liver (d), and LacZ-positive transplanted Rosa26 mouse hepatocytes (blue color) integrated in the liver of mouse 14 d (e) or 3 months (f) after intrasplenic transplantation. Note that transplanted cells did not proliferate over this time. (g, h) Ki67 in native hepatocytes (g) and bile duct cells (h) (dark nuclei) during liver regeneration after intraperitoneal cell transplantation alone. (i, j) Shows expression of Ataxia Telangiectasis Mutant gene (Atm) had become normal (i) and expression of p21 was no longer detected (j) in liver 7 d after transplantation of cells in peritoneal cavity alone. Orig. Mag., ×200, a, H&E; c-i, toluidine blue counterstain; j, eosin counterstain.

FIG. 5A-5H. Morphological properties of cells. Shown are phase contrast micrographs of (a) undifferentiated human embryonic stem cells (hESC), (b) hESC-derived meso-endoderm cells (hESC-MEC), (c) Epithelial cell adhesion molecule (Ep-CAM)-positive primary fetal liver epithelial stem/progenitor cells, FH-Ep-PP, and (d) EpCAM-positive fetal liver cells after three passages in cell culture (FH-Ep-P3) cells. Panels e-h show transmission electron microscopic images of undifferentiated hESC (e), hESC-MEC (f), primary FH-Ep-PP fetal cells, and cultured FH-Ep-P3 cells (h). The morphology of hESC differed markedly from other cell types with far less cytoplasmic complexity and general lack of multiple types of cell organelles, although hESC-MEC and fetal liver cells resembled one another with larger size and greater cytoplasmic complexity, including more mitrochondria, peroxisomes, lysosomes and vesicles. Orig. mag., a-d, ×600; e-h, ×2,500, size bars=1 μm.

FIG. 6. Outcomes in NOD.CB17-Prkdc^{scid}/J mice with induction of acute liver failure followed by treatment with hESC-MEC cell transplantation. Survival curves are shown in NOD/SCID mice with liver failure and treatments as indicated. All animals treated with hESC-MEC survived, whereas animals treated with epithelial cells from human cervix (Hela cells) and Sham-treated mice treated with vehicle alone showed mortality in 78-100%.

FIG. 7A-7D. Identification of transplanted hESC-MEC cells in recovered peritoneal tissue. Panel (A) shows hESC-MEC and HeLa cells along with mouse stromal cells in areas adjacent to microcarriers (mc) with hematoxylin staining of nuclei. In (B), expression of glucose-6-phosphatase (G-6-P), which is a property of hepatocytes, is shown by enzyme histochemistry in transplanted hESC-MEC (arrows), whereas HeLa cells did not express G-6-P. In (C), hESC-MEC show glycogen (arrows), whereas glycogen was absent in HeLa cells, consistent with their nonhepatic origin. In (D), in situ hybridization with pancentromeric human probe was combined with glycogen staining and this verified presence of transplanted cells, which are covered with multiple dark hybridization signals (arrows), along with glycogen in hESC-MEC cells. Orig. mag., ×600.

FIG. 8. Improved outcomes in NOD.CB17-Prkdc^{scid}/J mice with acute liver failure followed by transplantation of adult human hepatocytes, fetal human liver stem/progenitor cells, or hTERT-FH-B cells. Survival curves are shown in mice and treatments are indicated. hTERT-FH-B cells were originally isolated from normal human fetal liver followed by genetic modification to express the catalytic subunit of human telomerase reverse transcriptase gene, which increased their capacity to proliferate without altering their liver functions or stem/progenitor cell properties (Wege et al., 2003; Zalzman et al. 2003).

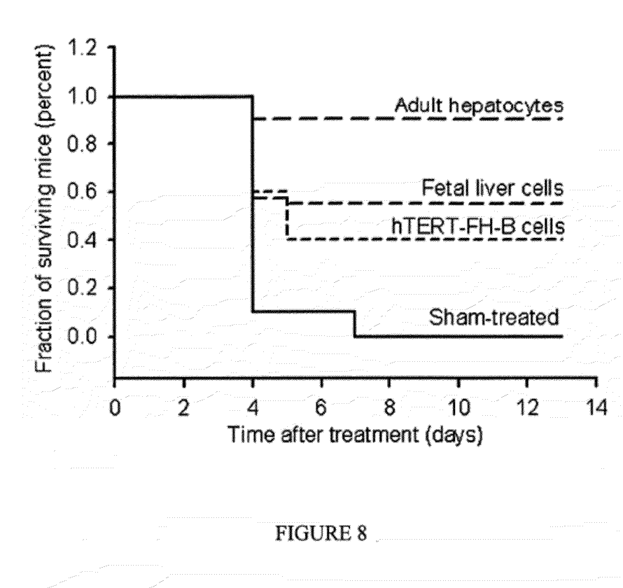

### DETAILED DESCRIPTION OF THE INVENTION

The present invention provides a method of treating liver failure in a subject comprising transplanting hepatocytes or stem cells or progenitor cells in an extrahepatic site in the subject in an amount sufficient to induce liver regeneration, wherein the transplanted hepatocytes or stem cells or progenitor cells are attached to extracellular matrix-coated microcarriers.

An additional reservoir of cells capable of offering liver support provides a method to treat subjects with liver failure in a nonacute setting.

The hepatocytes or stem cells or progenitor cells can be transplanted into any suitable extrahepatic site using methods known in the art. Preferred extrahepatic sites include the peritoneal cavity.

The transplanted cells can be, for example, mature hepatocytes, or fetal liver stem or progenitor cells, or cells derived from embryonic or equivalent stem or progenitor cells. The transplanted cells can be mesenchymal stem cells or stem cells with mesenchymal and epithelial phenotype. The transplanted cells can be stem cell derived cells, which can have a meso-endoderm phenotype. Cells of this meso-endoderm phenotype can be obtained by differentiation of cultured human embryonic stem cells or cells with equivalent stem cell potential or from the fetal human liver. The transplanted cells can be stem cell-derived liver-like cells that can be from multiple origins, including human embryonic stem cells (hESC), induced pluripotent stem (iPS) cells, or other types of stem cells.

As used herein, a microcarrier is a support matrix allowing for growth of adherent cells. Carrier materials for cells may be composed, for example, of gelatin, starch, porous glass, collagen or cellulose, or other materials. Well known brands of microcarriers include Cytodex® (Pharmacia Fine Chemicals AB) and Cultispher® (Percell Biolytica AB). Preferred microcarriers include Cytodex® 3 microcarriers (Amersham Biosciences Corp., Piscataway, N.J.). Preferred coatings for microcarriers include collagen. Preferrably, the extracellular matrix-coated microcarriers are biodegradable. Preferrably, the microcarriers are beads that are spherical in shape. The microcarriers can have dimensions of 100-6000 μm, preferably 100-1000 μm, more preferably 100-500 μm, and most preferably 100-300 μm.

The subject can be a subject with liver failure, in particular acute liver failure or chronic liver failure, ongoing liver failure or incipient liver failure, or a subject in danger of developing liver failure.

The invention also provides for the use of hepatocytes or stem cells or progenitor cells for preparation of a composition for transplantation in an extrahepatic site in a subject for inducing liver regeneration, wherein the hepatocytes or stem cells or progenitor cells are attached to extracellular matrix-coated microcarriers.

The invention provides for the use of the Ataxia Telangiectasia Mutant (ATM) gene signaling pathways in the diagnosis and recognition of molecular changes in drug-induced liver damage or liver damage arising from other possible toxins or mechanisms. This provides new methods for development of treatments capable of preventing deleterious changes in the ATM gene signaling pathways.

The invention is illustrated in the following Experimental Details section, which is set forth to aid in the understanding of the invention, and should not be construed to limit in any way the scope of the invention as defined in the claims that follow thereafter.

### EXPERIMENTAL DETAILS

### Example I

### Treatment of Liver Failure Using Hepatocytes

### Overview

A strain-specific model of acute liver failure in xenotolerant natural-onset diabetes severe combined immunodeficiency, NOD.CB17-Prkdc^{scid}/J mice, was used, where the antitubercular drug rifampicin (Rif), the anticonvulsant drug phenyloin (Phen), and the pyrrolizidine alkaloid monocrotaline (MCT), cause acute liver failure, and compared with immunocompetent mice in inbred C57/BL6 background, which suffer liver injury but not acute liver failure (Wu et al., 2008). As animals survived over several days, a window for cell therapy was available to establish whether extrahepatic support from transplanted hepatocytes would be sufficient for rescuing animals and whether proliferation of native hepatocytes could regenerate the liver without reseeding with healthy hepatocytes.

### Methods

Animals and procedures. The Animal Care and Use Committee of Albert Einstein College of Medicine approved animal use in conformity with National Research Council's Guide for the Care and Use of Laboratory Animals (United States Public Health Service publication, revised 1996). Male NOD.CB17-Prkdc^{scid}/J mice of 6-7 weeks age and C57BL/6J-^{Gtrosa26tm1Sor} (Rosa26) mice were from Jackson Labs (Bar Harbor, Me.). Fischer 344 (F344) rats were from the Special Animal Core of Marion Bessin Liver Research Center. Hepatocytes were isolated from donor F344 rats and Rosa26 mice with collagenase perfusion as described previously (Gupta et al., 2000; Wu et al., 2008). Cell viability was tested by trypan blue dye exclusion and was >80%. Mice were treated for 3 d with intraperitoneal (i.p.) rifampicin (Rif) (75 mg/kg) and phenyloin (Phen) (30 mg/kg) followed on day 4 by monocrotaline (MCT) (100-150 mg/kg). After 1 d, 10−50×10⁶ cells were transplanted i.p., with 1 ml Cytodex 3 microcarriers (Amersham Biosciences Corp., Piscataway, N.J.). Alternatively, 2×10⁶ cells were transplanted intrasplenically in addition (Wu et al., 2008). Cells were transplanted within 2 h after isolation. Multiple groups of animals were established, including mice treated with only vehicle and microcarriers. Animals were observed for encephalopathy, which was graded from 0 (absent) to 3 (coma), duration of survival, and mortality over 2 weeks. Some animals were sacrificed at intervals for studies (n=3-6). Drugs and chemicals were from Sigma Chemical Co. (St. Louis, Mo.).

Quantitative real-time polymerase chain reaction (qPCR). Total RNA was extracted by Trizol reagent (Invitrogen Corp., Carlsbad, Calif.), cleaned with RNeasy Kit and treated with DNAse (Qiagen Corp., Valencia, Calif.). cDNA was prepared from 1 μg total RNA with First Strand cDNA Synthesis Kit (C-01, SA Biosciences, Frederick, Md.). Expression of 84 relevant genes was analyzed by RT² Real-Time SYBR Green PCR Array for Mouse Stress and Toxicity Pathway Finder (PAMM-003, SA Biosciences) in ABI 7000 instrument (Applied Biosystems, Foster City, Calif.). Amplifications were in 25 μl with denaturation for 10 min at 95° C., followed by 40 cycles at 95° C.×15 s, annealing×60 s at 60° C. Each condition was in triplicate. Gene expression was normalized to β-actin. Threshold cycle (Ct) values were determined with ABI Prism 7000 SDS software. Fold-changes in gene expression were determined by 2̂(−ΔΔCt) method with SA Biosciences software.

Histological studies. Liver samples were fixed in 10% formalin for paraffin embedding and sections were stained with hematoxylin and eosin. To identify transplanted cells, tissues were frozen in methylbutane to −80° C. Cryosections of 5 μm were fixed in ethanol for 10 min. LacZ, glycogen and glucose-6-phosphatase (G-6-P) were stained histochemically, as described previously (Gupta et al., 2000; Rajvanshi et al., 1996). Additional stainings were for cyclin-dependent kinase inhibitor 1A (p21) (1:50, sc-397, Santa Cruz Biotechnology Inc., Santa Cruz, Calif.), proliferation-related Ki-67 antigen (Ki67) (1:1000, VP-K451, Vector Labs Inc., Burlingame, Calif.), heme oxygenase 1 (Hmox1) (1:50, AB128, Chemicon International Inc., Temecula, Calif.), glutathione-S-transferase (Gstm1) (1:200, primary antibody in rabbit from I. Listowski, Albert Einstein College of Medicine), ataxia telangiectasia mutant (ATM) (1:150, AB370, Chemicon), and 8-oxo-dG (4359-MC-100, Trevigen Inc., Gaithersburg, Md.). Peroxidase-conjugated goat anti-mouse or anti-rabbit immunoglobulins (1:500=−600 of #3682 and #AO545, respectively, Sigma) were used with diaminobenzidine kit (K3465, Dako Corp., Carpinteria, Calif.). For negative controls, primary antibodies were omitted. For Ki67, positive controls were 40 h after two-thirds partial hepatectomy. Apoptosis was shown with ApopTag Peroxidase In Situ Kit (Chemicon).

Serological assays. Serum was stored at −20° C. and analyzed for ALT, alkaline phosphatase, and total bilirubin with automated clinical system. Prothrombin time was measured with Plastinex thromboplastin (101158, Bio/Data Corp., Horsham, Pa.). Plasma was separated from blood collected in 0.11 M sodium citrate and diluted 7:3 with water. To 0.1 ml plasma, 0.2 ml reconstituted in water and preincubated with plasma at 37° C. for 5 min was added and time to clotting was measured.

Statistical methods. Data are shown as means±SD. Significances were analyzed by t-tests, Chi-square test or ANOVA and Holm-Sidak pairwise comparisons with SigmaStat 3 (Systat Software Inc., Point Richmond, Calif.). P values <0.05 were considered significant.

### Results

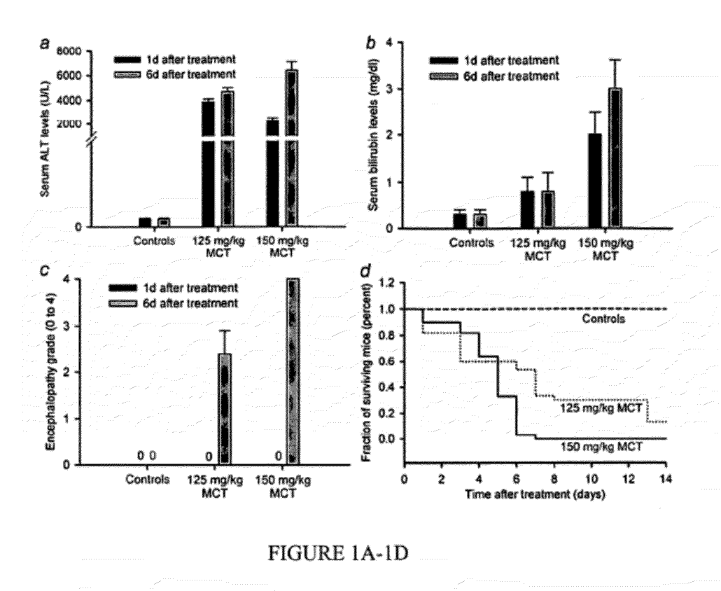

Liver injury and mortality in mice exposed to drugs. Rifampicin (Rif), phenyloin (Phen) and monocrotaline (MCT) caused extensive liver injury in NOD.CB17-Prkdc^{scid}/J mice. This manifested shortly after priming over 3 d with 75 mg/kg Rif plus 30 mg/kg Phen daily followed by one dose of either 125 or 150 mg/kg MCT. By contrast, NOD.CB17-Prkdc^{scid}/J mice tolerated Rif, Phen and 100 mg/kg MCT without overt hepatotoxicity. Further studies were performed in mice treated with vehicle alone (n=10), Rif, Phen and 125 mg/kg MCT (n=30), and Rif, Phen and 150 mg/kg MCT (n=30). Serum alanine aminotransferase (ALT) and total bilirubin, indicators of liver injury, rose significantly in drug-treated mice (FIGS. 1a and 1b). Mean serum ALT was 58±3, 4689±300, and 6380±689 units/L, and bilirubin was 0.3±0.1, 08±0.4, and 3.0±1.0 mg/dl, in control mice and mice 3-6 d after Rif, Phen and 125 mg/kg or 150 mg/kg MCT, respectively (p<0.05, ANOVA with Holm-Sidak test). Prothrombin time was prolonged, which reflected severe liver injury. Virtually all mice developed encephalopathy 6 d after drug treatments and this was worse in mice given 150 mg/kg MCT (FIG. 1c). Extensive mortalities were observed in drug-treated mice (FIG. 1d). After Rif, Phen and 150 mg/kg MCT, 50% of mice were dead in 5.5 d and 100% in 7 d. Duration of survival was slightly longer after Rif, Phen and 125 mg/kg MCT, with 40% dying after 5.5 d, 67% after 7 d, and 87% after 14 d (p=n.s.). By contrast, vehicle-treated control mice remained alive and well (p<0.05, logrank tests). In view of relatively longer survival of mice, the dose of 125 mg/kg MCT was chosen for additional studies.

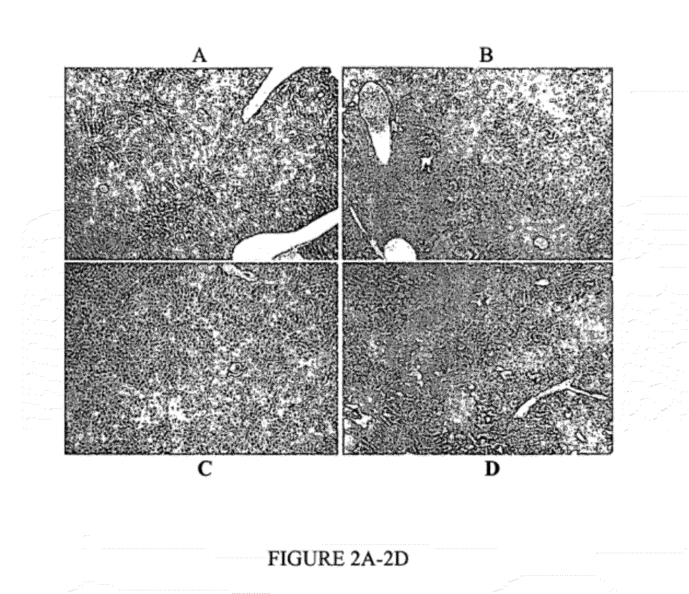

Tissue studies from drug-treated mice showed extensive liver necrosis. The liver of control mice was normal (FIG. 2A). By contrast, 1 d after Rif, Phen and 125 mg/kg MCT, 30-40% of the liver parenchyma was destroyed (FIG. 2B). This injury was repaired and parenchymal integrity seemed temporarily restored after 3 d (FIG. 2C). However, liver injury resumed, such that after another 4 d, 50-70% of the liver parenchyma was lost (FIG. 2D).

During this injury, the liver was not infiltrated with inflammatory cells, e.g., neutrophils, macrophages, eosinophils or basophils. It should be noteworthy that NOD.CB17-Prkdc^{scid}/J mice lacked T and B lymphocytes. The time-course of tissue changes was in agreement with the kinetics of abnormalities in liver tests, prothrombin time, encephalopathy, and mortality. Moreover, proliferation of bile duct cells or other liver cell subpopulations was not observed.

Molecular basis of liver injury in drug-treated NOD.CB17-Prkdc^{scid}/J mice. To determine the effects of Rif, Phen and MCT on liver cell turnover, hepatic apoptosis and cell proliferation rates were studied in multiple tissue sections. Only slightly more apoptotic cells were observed in drug-treated mice, 5-8 cells per section, versus 0-1 cells per section in control mice, indicating tissue necrosis constituted the predominant mode of injury. In mice with liver damage, hepatocytes in periportal areas expressed Ki67, the proliferation marker, especially 6-8 d after drugs. Cell proliferation activity was far greater than control animals, although much less than after liver regeneration induced by two-thirds partial hepatectomy. This suggested that some hepatocytes in the damaged liver could potentially have traversed the cell cycle, although this was restricted by other changes, and failure of liver regeneration obviously accounted for death in animals.

Administration of Rif and Phen for 3 d increased hepatic expression of Cyp3a4 enzyme, which metabolizes MCT (Wu et al., 2008), and directed drug toxicity. This Cyp3a4 induction was similar in C57BL/6J mice. However, the latter mice did not develop liver injury seen in NOD.CB17-Prkdc^{scid}/J mice, incriminating additional mechanisms (Wu et al., 2008).

An array of 84 genes was examined by quantitative reverse-transcription polymerase chain reactions (qPCR) for mRNA expression. These genes represented hepatic stress and toxicity pathways, including oxidative/metabolic stress, cell proliferation, growth arrest, inflammation, DNA damage and repair (Table 1). Expression of 35%-44% of the 84 genes changed >2-fold in mice 1, 3 and 7 d after Rif, Phen and 125 mg/kg MCT, compared with control mice (Table 2). Categorization of these genes indicated significant oxidative/metabolic stress with upregulation of glutathione peroxidase, glutathione-S-transferase mu (Gstm), heme oxygenase, Cyp450 genes, and heat shock proteins. Many Cyp450 genes were downregulated, consistent with loss of perivenous hepatocytes, which expressed these genes. Expression of genes related to inflammation or apoptosis was not prominently altered, which was in agreement with tissue studies. However, critical regulators of DNA damage and repair, cell proliferation, and cell growth arrest were abnormally expressed. In particular, Atm was expressed less, whereas p21 was expressed at extremely high levels. In follow-up studies at the protein level, the onset of oxidative/metabolic stress, DNA damage and cell growth arrest was verified by immunostaining of liver sections. Findings included increased expression of Gstm1, oxidative DNA adducts on guanosine residues (8-oxo-dG), decreased Atm expression and increased p21 expression. These changes were observed 1 d after Rif, Phen and MCT and persisted subsequently until death.

The confluence of drug-induced hepatic injury in the setting of early decreases in Atm expression, along with changes in Atm-associated DNA damage and repair, and cell growth arrest through regulation by p21 and transformed mouse 3T3 cell double minute (Mdm2), suggested that Atm signalling was perturbed. This was verified by plotting of Atm signalling pathways, which implicated perturbations in key limbs of these pathways after Rif, Phen and MCT-induced injury. In control animals, Atm was expressed normally, along with several other pathway members. By contrast, greater expression of Mdm2 and p21, particularly of p21 throughout the period of liver injury, and of Growth arrest and DNA-damage-inducible 45 alpha (Gadd45a) transiently during the liver injury, indicated that DNA damage in hepatocytes was responsible for activating cell cycle checkpoint controls to arrest cell growth and cell cycling. These changes applied to surviving liver cells, as necrotic cells had largely been removed from the liver at later times.

It was relevant to determine whether provision of suitable liver support would provide sufficient time for liver regeneration with reversal of cell growth-arrest induced by oxidative DNA damage and p21 overexpression. Alternatively, defining whether reseeding of the damaged liver with healthy cells will be a mandatory requirement to advance liver regeneration during rescue of animals with liver failure was appropriate.

Mechanisms in rescue of mice with liver failure by cell therapy. To provide extrahepatic support in xenotolerant NOD.CB17-Prkdc^{scid}/J mice with liver failure, hepatocytes were transplanted with collagen-coated Cytodex3 microcarriers, which promoted vascularization of transplanted cells in the peritoneal cavity. As hepatocytes could be transplanted in large numbers, equaling or exceeding those in the liver, the benefit of graded hepatic support from transplanted cells was examined. It is noteworthy that transplanted hepatocytes do not migrate from the peritoneal cavity to other organs, e.g., the liver. Rif, Phen and 125 mg/kg MCT were administered followed 2 d later by intraperitoneal injection of microcarriers alone (n=10), or 5, 10 and 50×10⁶ freshly isolated F344 rat hepatocytes, along with microcarriers (n=10-20). This represented transplantation of 5%, 10%, and 50% of the hepatocyte mass in healthy mouse liver.

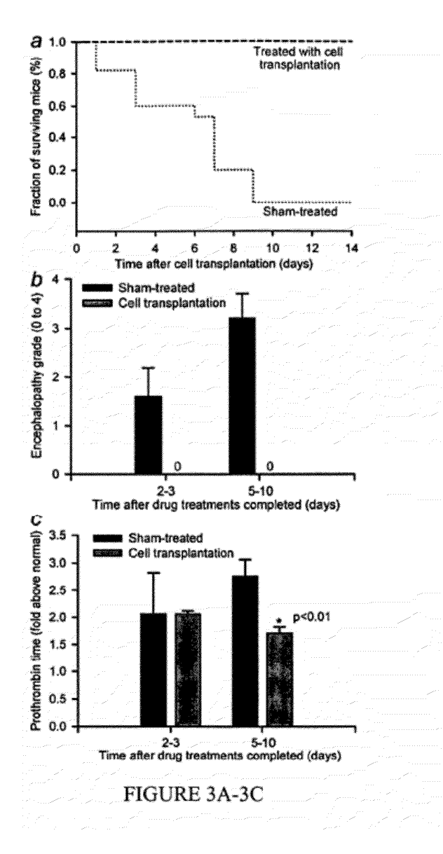

All mice treated with microcarriers alone died within 2 weeks, whereas mice treated with cells survived, including after transplantation of only 5×10⁶ cells (p<0.001, logrank test) (FIG. 3a). Mice treated with cells remained healthy, active and without encephalopathy, although mice treated with microcarriers alone showed progressive encephalopathy (FIG. 3b). In mice treated with cells, prothrombin times were lower 5-10 d after drugs (p<0.01), compared with mice treated with microcarriers alone (FIG. 3c). These findings indicated that extrahepatic support from cells rescued mice with liver failure.

In another group of NOD.CB17-Prkdc^{scid}/J mice with liver failure, medium harvested from hepatocytes cultured in serum-free conditions for 2 d was injected once daily into the peritoneal cavity. This did not alter mortality, indicating that transplantation of hepatocytes was necessary for rescuing animals with liver failure.

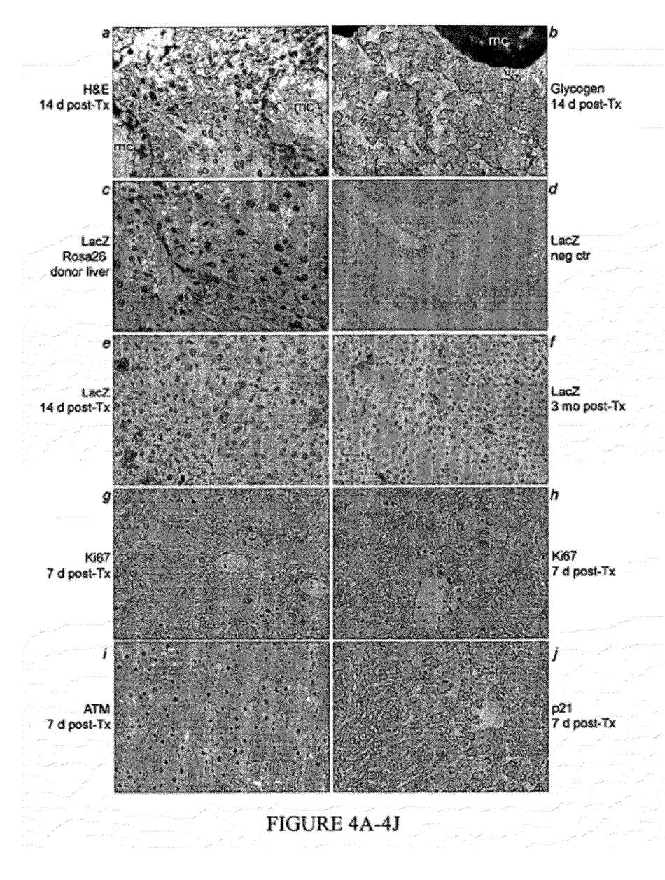

Verification was performed of the integrity of transplanted hepatocytes and presence of hepatic functions in cell-microcarrier conglomerates recovered from mice (FIGS. 4a, 4b). Transplanted hepatocytes were morphologically intact and contained glycogen, as well as glucose-6-phophatase (G-6-P), which are critical for glucose metabolism. To address whether the liver could regenerate after cell transplantation in the peritoneal cavity without requiring reseeding with healthy cells, additional mice were treated with Rif, Phen and 125 mg/kg MCT (n=15). After 2 d, 2×10⁶ Rosa26 donor hepatocytes expressing E. coli β-galactosidase (LacZ) gene were transplanted in the liver via the spleen and 5×10⁶ F344 rat hepatocytes were simultaneously transplanted into the peritoneal cavity. Again, all mice treated with cell transplantation survived for at least 14 d. Transplanted hepatocytes were integrated in the liver parenchyma, as was expected, with 2-4 transplanted cells per liver lobule (FIG. 4c-4f). The number of transplanted cells in the liver did not change between 2, 4 and 12 weeks after cell transplantation, indicating absence of proliferation in transplanted hepatocytes. On the other hand, Ki67 staining showed hepatocytes proliferating in the liver and proliferating cells were also observed in bile ducts (FIG. 4g-4h), which is consistent with liver regeneration from native liver cells. In these cells, hepatic expression of Atm returned to normal and p21 was no longer detected by immunostaining (FIG. 4i-4j). Tissue analysis verified that liver morphology remained normal after cell therapy throughout the 12 week period of the studies.

Analysis of livers with qPCR arrays in mice 7 d after intraperitoneal cell transplantation showed only 2 genes were differentially expressed, Bax and p21, compared with differential expression of 28 genes previously (Table 2). Moreover, these genes were expressed at far lower levels compared with mice without cell therapy, 2-fold (Bax) and 10-fold (p21) lower, indicating substantial correction of the molecular abnormalities observed.

TABLE 1
List of Genes in PAMM-003 Array.

Oxidative or Metabolic Stress: Cryab (a-Crystallin B), Cyp1a1, Cyp1b1,
Cyp2a5, Cyp2b9, Cyp2b10, Cyp2c29, Cyp3a11, Cyp4a10, Cyp4a14,
Cyp7a1, Ephx2, Fmo1, Fmo4, Fmo5, Gpx1 (glutathione peroxidase),
Gpx2, Gsr (glutathione reductase), Gstm1 (glutathione S-transferase
mu1), Gstm3, Hmox1, Hmox2, Mt2, Polr2k (Mt1a), Por, Sod1, Sod2.
Heat Shock: Dnaja1, Hsf1 (tcf5), Hspa1b (Hsp70-1), Hspa11 (hsp70 11),
Hspa4 (hsp70), Hspa5 (grp78), Hspa8, Hspb1 (Hsp25), Hspd1 (Hsp60),
Hspe1 (chaperonin 10).
Proliferation and Carcinogenesis: Ccnc (cyclin C), Ccnd1 (cyclin D1),
Ccng1 (cyclin G), E2f1, Egr1, Pcna.
Growth Arrest and Senescence: Cdkn1a (p21Waf1/p21Cip1), Ddit3
(GADD153/CHOP), Gadd45a, Igfbp6, Mdm2, Trp53 (p53).
Inflammation: Ccl21b, Ccl3 (MIP-1a), Ccl4 (MIP-1b), Csf2 (GM-CSF),
Cxcl10 (Scyb10), Il1a, Il1b, Il6, Il18, Lta (TNF b), Mif, Nfkb1, Nos2
(iNOS), Serpine1 (PAI-1).
DNA Damage and Repair: Atm, CHK2 checkpoint homolog (Chek2),
Ercc1, Ercc4, Rad23a, Rad50, Ugt1a2, Ung, Xrcc1, Xrcc2, Xrcc4.
Apoptosis Signaling: Anxa5 (annexin v), Bax, Bcl2l1 (bcl-x), Casp1
(Caspase1/ICE), Casp8 (caspase8/FLICE), Fasl (Tnfsf6), Nfkbia
(ikBa/Mad3), Tnfrsf1a (TNFR1), Tnfsf10 (TRAIL), Tradd.

TABLE 2
Cumulative changes in expression of 84 genes in
PAMM-003 array analysis.

| | 1 d after | 3 d after | 7 d after | P < 0.05 |
|---|---|---|---|---|
| Description | drugs | drugs | drugs | (χ² test) |
| Total number genes | 84 | 84 | 84 | — |
| analyzed | (100%) | (100%) | (100%) | |
| (%) | | | | |
| Number genes | 30 | 37 | 28 | No |
| expressed | (36%) | (44%) | (33%) | |
| differently | | | | |
| (%) | | | | |
| Number genes | 12 | 18 | 11 | No |
| upregulated by 2- | (14%) | (21%) | (13%) | |
| fold or greater | | | | |
| (%) | | | | |
| Number genes | 18 | 19 | 17 | No |
| downregulated by 2- | (21%) | (23%) | (20%) | |
| fold or greater | | | | |
| (%) | | | | |

Based on 2-fold or greater change versus controls with p < 0.05 in group comparisons.

### Discussion

Extensive liver necrosis in the Rif, Phen and MCT model in NOD.CB17-Prkdc^{scid}/J mice reproduced key aspects of acute liver failure. Drug toxicity is a major cause of liver failure in people. Acetaminophen is the commonest offender, although Rif, Phen, as well as pyrrolizidine alkaloids are incriminated (Murray et al., 2008). The broad window of therapeutic opportunity in this model allowed delineation of the benefits of hepatic or extrahepatic transplantation of cells. The ability to promote recovery of the damaged liver, or to provide liver support, by extrahepatic cell transplantation alone as disclosed herein should be clinically very significant.

The present studies also advance the molecular basis of liver injury produced by drug-induced oxidative/metabolic stress and genotoxicity. In particular, the significance of Atm pathways in acute liver failure has been unknown. Previously, the Mad transcription factor, which dominantly antagonizes the activity of c-Myc (myelocytomatosis) protooncogene, was shown to induce liver failure through a discrete genetic mechanism, although the predominant mode of liver injury in that setting was apoptotic (Gagandeep et al., 2000). Toxicity through transgenes, e.g., prodrug-activating herpes simplex virus thymidine kinase (HSV-TK) produced liver injury, although HSV-TK-induced damage differed from typical consequences of liver failure (Braun et al., 2000). Transplanted hepatocytes did proliferate in the liver after Mad- or HSV-TK-induced liver injury. Similarly, transplanted hepatocytes showed limited proliferation in the liver after D-galactosamine-induced injury (Gupta et al., 2000). However, the molecular basis of liver injury in those animal models was not fully established. And it was unknown whether extrahepatic support could have permitted recovery and proliferation of native hepatocytes.

A major aspect of liver injury produced by Rif, Phen and MCT concerned extensive oxidative/metabolic stress in the liver, as reflected by increased expression of Gpx2, Gstm3, and Hmox1 in the present sampling. Although hepatic stress often regulates cytochrome P450 genes, many of these genes were expressed at very low or undetectable levels, likely due to the loss of perivenous hepatocytes, where these genes are normally expressed. The present gene array studies were in agreement with complex cellular events after drugs. Besides the ability of Rif and Phen to promote conversion of MCT to reactive metabolites capable of hepatic injury and DNA adduct formation, these drugs may damage DNA through alternative mechanisms (Bhuller et al., 2006; Chowdhury et al., 2006; Wang et al., 2005; Wu et al., 2008). For instance, Phen has been shown to be genotoxic in the setting of Atm deficiency (Bhuller et al., 2006). While the role of Atm deficiency in susceptibility to liver injury is undefined, in Atm−/− mice partial hepatectomy-induced liver regeneration was impaired (Lu et al., 2005). In other studies, the remnant liver after partial hepatectomy exhibited oxidative stress, 8-oxo-dG DNA adducts, p21 expression, and decreased hepatocyte proliferation (Gorla et al., 2001; Sigal et al., 1996). This offers insights into how Atm deficiency could impair liver regeneration, and will also be in agreement with the pathophysiological role of Atm signaling in Rif, Phen and MCT-induced liver failure. The role of Atm as master regulator of the cellular response to DNA breaks and ensuing cell cycling, including through phosphorylation of multiple effectors, is well established (Kanu and Behrens, 2008). After DNA damage, Atm, along with other genes, e.g., Chek1 and Chek2, phosphorylates p53. In turn, p53 would activate p21, leading to the arrest of damaged cells in G1/S through corresponding cyclins and cyclin-dependent kinases (Kuribayashi and El-Deiry, 2008). In the present studies, early and persistent downregulation of Atm was observed after Rif, Phen and MCT, where extensive oxidative/metabolic stress had been initiated and DNA repair mechanisms were overwhelmed, as indicated by downregulation of Rad50 and Xrcc4 genes. This was associated with marked upregulation of p21 expression with G1/S checkpoint arrest, since Cyclin g1 (Ccng1) was upregulated, while Cyclin d1 (Cccnd1), Egr1 and E2F were up- or downregulated at various times. Upregulation of Gadd45a, Ddit3, and Mdm2 provided more evidence for p53-regulated cell growth arrest (Zhang and Chen, 2008). These changes were reflected by only few hepatocytes with Ki67, which is characteristic of cells in late G1, S and G2/M. As cell therapy normalized these genetic changes, except for expression of p21 at lower levels, and damaged hepatocytes could replicate without reseeding with healthy hepatocytes, this will be in agreement with clinical observations showing that the liver may recover totally despite extensive damage and acute failure (Quaglia, 2008). It was noteworthy that transplantation of cells was required for improving outcomes in liver failure since intraperitoneal injection of conditioned medium from hepatocytes alone was ineffective in the present studies. Xenogeneic rat hepatocytes were transplanted for convenience in the present studies because far more donor mice would otherwise have been required for these studies.

The present studies differed from previous studies in other respects, including incorporation of reporter hepatocytes to determine whether native hepatocytes could recover and regenerate the liver. Although some patients with acute liver failure have been treated with transplantation of hepatocytes in the peritoneal cavity (Habibullah et al., 1994), those studies did not include extracellular matrix support, without which hepatocytes are rapidly destroyed. Numerous cell transplantation studies have been performed in animals with liver failure, but lack of insights in the fate of transplanted cells, mechanisms in liver injury and repair, initiation of therapy before induction of liver injury, short therapeutic windows, and other confounding issues, make it difficult to interpret those studies. By contrast, incorporation of microcarriers permitted revascularization of transplanted cells, which was beneficial for transplanted cell survival and function.

As cell therapy was effective even when cells representing only five to ten percent of the liver mass were transplanted, this should be clinically feasible. Transplantation of cells in the peritoneal cavity will be far simpler than intravascular injection of cells in the portal bed in acute liver failure with attendant coagulopathies. The microcarriers used in the present studies were biodegradable and did not cause intra-abdominal lesions, intestinal obstruction or fibrosis. Clinical applications should be facilitated by identification of donor cells other than from human liver, e.g., stem cells are of interest for that purpose. Acute liver failure in NOD.CB17-Prkdc^{scid}/J mice will be especially helpful for defining the therapeutic potential of stem cell-derived cells, since these mice tolerate human xenografts, including in the peritoneal cavity (Cho et al., 2004).

### Example II

### Treatment of Liver Failure Using Cells Derived from Human Embryonic Stem Cells, Adult Human Liver and Fetal Human Liver

### Introduction and Overview

During embryonic and fetal development, pluripotential stem cells originate organ-specific stem/progenitor cells. In turn, these stem/progenitor cells produce cell lineages in adult organs, e.g., hepatocytes, the major cell-type of the liver, arise from fetal hepatoblasts. Recently, human embryonic stem cells (hESC), derived from the inner cell mass of the embryo at the blastocyst stage (Thomson et al., 1998), and iPS, derived by nuclear reprogramming of somatic cells from individuals (e.g., Nakagawa et al., 2008), gathered major interest for cell therapy and other applications. Stem cells are largely lacking in hepatic markers, whereas fetal liver stem/progenitor cells exhibit multilineage patterns of gene expression, and mature hepatocytes express characteristic complements of hepatic genes (Inada et al., 2008a,b). Also, fetal human liver cells immortalized by the catalytic subunit of human telomerase reverse transcriptase (hTERT-FH-B cells) were found to retain extensive proliferation capacity and stem cell properties (Wege et al., 2003; Zalzman et al., 2003). This interconnected framework of molecular genetics and cellular phenotypes offers opportunities for identifying intermediate stages of cells during hepatic lineage progression, as well as methods for cell therapy.

Recently, fetal human liver stem/progenitor cells were shown to display unique conjoint meso-endoderm phenotype with the ability to generate mesoderm lineages, e.g., adipocytes, osteocytes, endothelial cells, as well as endoderm lineages, e.g., hepatocytes (Inada et al., 2008b). Remarkably, fetal liver cells regressed in vitro with greater expression of mesoderm genes, e.g., vimentin or α-smooth muscle actin (SMA), and lesser expression of endoderm/epithelial genes, e.g., E-cadherin, albumin (Alb), glucose-6-phosphatase (G-6-P), glycogen, cytokeratin (CK)-19, γ-glutamyltranspeptidase (GGT), dipeptidyl peptidase IV (DPPIV), under transcriptionally defined settings, such as increased expression of the endoderm-specifying transcription factor, FoxA2. This led to the consideration that if the conjoint meso-endodermal stage in fetal liver cells were obligatory for differentiation of hepatocytes, such a phenotype should be observed during differentiation of stem cells. Therefore, the present study was carried out with putative mesenchymal stem cells arising spontaneously in cultured WA01 hESC (WiCell Research Institute, Madison, Wis.) with the potential to differentiate into mesoderm (adipocytes, bone, cartilage or blood cells) (Olivier et al., 2006). However, in the present study, these hESC-derived cells were found to possess properties of mesenchymal as well as epithelial cells, including hepatic properties, which is consistent with a meso-endoderm phenotype; these cells were designated hESC-MEC. In the present study, similarities in hESC-derived hESC-MEC cells and cultured fetal human hepatocytes are reported, as well as the potential of these relatively immature liver cells for supplementing deficient hepatic functions in a mouse model of toxic drug-induced acute liver failure (ALF), where native hepatocytes retained the capacity for regenerating the liver without requiring hepatic reseeding with healthy transplanted hepatocytes. Moreover, in the present study, the efficacy was demonstrated for cell therapy in ALF of fetal human liver cells, including (hTERT-FH-B cells) (Wege et al., 2003), and of adult human hepatocytes isolated from donor organs that had not been used for the purpose of liver transplantation in people.

### Materials and Methods

Institutional approvals. The Committee on Clinical Investigations, Embryonic Stem cell Oversight Committee (ESCRO) and Animal Care and Use Committee (ACUC) reviewed and approved protocols.

hEScells and cell culture. hESC were cultured on irradiated feeder cells in DMEM/F12 medium, 20% Knock-out Serum Replacer (KSR), 2 mM L-glutamine, 0.1 mM MEM Non Essential Amino Acids Solution (NEAA), 1% penicillin-streptomycin (Invitrogen Corp., Carlsbad, Calif.), and 4 ng/ml basic fibroblast growth factor (R&D Systems, Minneapolis, Minn.) (complete medium). Cells were passaged each week. hESC-MEC cells were obtained by spontaneous differentiation and were cultured in DMEM with 10% FBS (Olivier et al., 2006). For conditioned medium, hESC-MEC were cultured for 24 h in complete medium followed by in DMEM for 24 h, which was harvested and passed through a 0.22 μm filter (Millipore, Billerica, Mass.).

Fetal human liver cells and adult human hepatocytes. Fetal livers of 19-24 week gestation were from Human Fetal Tissue Repository, Albert Einstein College of Medicine. Ep-CAM-positive cells were isolated by immunomagnetic beads and cultured as described previously (Inada et al., 2008b). Adult hepatocytes were from ADMET Technologies Inc. (Durham, N.C.). These cells were isolated from unused donor livers (H0852-P10a, H0852-P15, and H0796-U10) by collagenase digestion followed and the cell viability before cryopreservation was 76% to 82%. Cells were cryopreserved as described previously (Inada et al., 2008b, Cho et al., 2004). For transplantation studies, fetal liver tissue was digested with collagenase for 20 to 30 min at 37° C. and cells were passed through 80 μm dacron, pelleted under 350×g for 5 min at 4° C., and resuspended in DMEM (Invitrogen, Carlsbad, Calif.) for cryopreservation. For transplantation, frozen cells were rapidly thawed to 37° C. Cell number and viability was determined in Neubauer hemocytometer with exclusion of 0.2% trypan blue dye. Immortalized hTERT-FH-B cells were cultured in DMEM medium with 10% FBS, 100 U/ml penicillin, 100 μg/ml streptomycin, 5 μM hydrocortisone, and 5 μg/ml insulin at 37° C. in humidified atmosphere containing 5% CO2 in room air. Cells were subpassaged at a ratio of 1:3 every 3 to 5 d with trypsin-EDTA for 2 min at 37° C. For transplantation, cells were released from culture dishes by trypsin-EDTA followed by mixing with Cytodex3 microcarrier beads.

Differentiation of hESC-MEC. To induce osteogenic and adipogenic differentiation, cells were cultured in DMEM with 10% FBS and additives for 3 weeks as described previously (Inada et al., 2008b; Olivier et al., 2006). Cells were cultured on fibronectin (Sigma Chemical Co., St. Louis, Mo.) for endothelial differentiation (Ria et al., 2008). For endoderm differentiation, cells were cultured in RPMI 1640 medium without serum for 2 d, 0.2% serum for 2 d, and 2% serum for 2 weeks with activin A (100 ng/ml), a-FGF (100 ng/ml), HGF (20 ng/ml), OSM (20 ng/ml), DKK-1 (20 ng/ml) (R&D Systems), trichostatin A (100 nM/ml), and γ-secretase inhibitor X (Calbiochem, La Jolla, Calif.).

Immunohistochemistry. Cells were fixed in 4% paraformaldehyde in PBS (PAF) and blocked/permeabilized 0.2% Triton X-100 and 5% goat serum (Sigma) in PBS for 1 h. Overnight incubations at 4° C. were with anti-human mouse antibodies: anti-Oct¾ (1:200; Santa Cruz Biotechnology Inc., Santa Cruz, Calif.), anti-SSEA4 (1:50; R&D Systems), anti-TRA-1-60 (1:50; Chemicon International Inc., Temicula, Calif.). After washing with PBS, cells were incubated for 1 h with TRITC-conjugated goat anti-mouse IgG (1:50, Sigma) and counterstained with 4′-6-diamidino-2-phenylindole (DAPI) (Invitrogen). In negative controls, primary antibodies were omitted. Glycogen, G-6-P, GGT, and DPPIV were stained as previously described (Inada et al., 2008b).

Electron microscopy. Cells were fixed in 2.5% glutaraldehyde in cacodylate butter, postfixed in osmium tetroxide and stained with 1% uranyl acetate before plastic embedding. Ultrathin sections were examined under JEOL 1200 electron microscope.

Molecular studies. RNA was extracted with TRIzol reagent (Invitrogen), cleaned by RNeasy (Qiagen Sciences, Germantown, Md.), incubated with DNase I (Invitrogen) and reverse-transcribed with Omniscript RT kit (Qiagen). Platinum PCR SuperMix (Invitrogen) was used for PCR with annealing at 94° C.×5 min, and 35 cycles at 94° C.×30 s, 55° C.×30 s, 72° C.×45 s, and 72° C.×10 min. Primers are listed in Table 3. Mouse Stress and Toxicity RT² Profiler PCR Array and RT² Real-Time SyBR Green PCR mix and RT² First Strand kit were from SuperArray Biosciences (Frederick, Md.). cDNA synthesis and PCR were according to the manufacturer instructions. For data analysis, ΔΔCt method was used. Fold-changes in gene expression were expressed as log-normalized ratios from sham-treated/normal and cell transplantation/normal livers. Cellular gene expression was analyzed with U133 2.0 Plus oligonucleotide arrays (Affymetrix Corp., Santa Clara, Calif.) (Inada et al., 2008b). Differentially expressed genes were analyzed with SAS software (SAS Institute Inc., Cary, N.C.). Gene lists were annotated and categorized with DAVID package (National Institute of Allergy and Infectious Disease, Bethesda, Md.) and gene ontology groups or pathways were according to Kyoto Encyclopedia of Genes and Genomes (KEGG). Representation of specific pathways in differentially expressed genes was determined with PathwayStudio 5.0 (Aridane Genomics, Rockville, Md.).

Gene transfer. Lentivirus vector (LV) expressing green fluorescent protein (GFP) under phosphoglycerate kinase (PGK), mouse albumin (Alb) enhancer-promoter, transthyretrin (TTR), and α-1-antitrypsin (AAT) promoters were prepared (Inada et al., 2008b). Cells were transduced with LVs at multiplicity of infection (MOI) of 10. GFP expression was analyzed after 4 d by fluorescence microscopy and flow cytometry.

Natural-onset diabetes severe combined immunodeficiency (NOD/SCID) mice with liver failure. CB17.NOD/SCID^{prkdc} mice, 6-7 weeks old, were obtained from Jackson Labs. (Bar Harbor, Me.). Mice were given 3 daily doses of i.p. Rif (75 mg/kg) and Phen (30 mg/kg) followed by one i.p. dose on day 4 of MCT. After 1 d, 4-6×10⁶ cells were transplanted i.p. with 1 ml Cytodex 3™ microcarriers (Amersham Biosciences Corp., Piscataway, N.J.). Sham-treated animals received vehicle and microcarriers. Encephalopathy was graded from 0 (absent) to 3 (coma). Animals were observed for 2 weeks. In other mice, 1×10⁶ hESC-MEC were injected into the portal vein. These animals were sacrificed 5 d after cell transplantation. Transplanted cells were identified by DNA PCR for SRY and in situ hybridization for alphoid satellite sequences in centromeres (Gupta et al., 1994). For hepatic function in transplanted cells, glycogen and G-6-P were stained (Nakagawa et al., 2008). To visualize GFP in hESC-MEC transduced with LV-Alb-GFP, tissues were fixed in 4% PAF, equilibrated in 20% sucrose and frozen in methylbutane at −80° C. followed by immunostaining with rabbit anti-GFP (1:300, Molecular Probes) (Inada et al., 2008b). Sections were incubated with FITC-conjugated goat anti-rabbit IgG and counterstained with DAPI. For Ki67, tissues were fixed in 4% PAF followed by rabbit anti-Ki67 (1:750, Vector Laboratories, Burlingame, Calif.) and secondary anti-Rabbit Alexa Fluor 546 (1:500, Molecular Probes).

Serum human albumin. Stored sera were analyzed with human albumin ELISA quantitation Kit (Bethyl Laboratories) according to manufacturer's instructions.

Human cytokine array. Cytokines in conditioned medium were detected by biotin label-based human antibody array I membrane for 507 human proteins according to the manufacturer's instructions (RayBiotech, Norcross, Ga.).

Statistical analysis. Data were analyzed by t-tests, logrank tests, and ANOVA with Holm-Sidak posthoc test. P values <0.05 were considered significant.

TABLE 3
Oligonucleotide primers for RT-PCR

| | | Amplicon | SEQ |
|---|---|---|---|
| | | size | ID |
| Gene | Primer sequence 5′-3′ | expected | NO: |
| Oct4 | F: GACAACAATGAAAATCTTCAGGAGA | 218 bp | 1 |
| | R: TTCTGGCGCCGGTTACAGAACCA | | 2 |
| Alb | F: TGCTTGAATGTGCTGATGACAGGG | 161 bp | 3 |
| | R: AAGGCAAGTCAGCAGGCATCTCATC | | 4 |
| AFP | F: TGCAGCCAAAGTGAAGAGGGAAGA | 260 bp | 5 |
| | R: CATAGCGAGCAGCCCAAAGAAGAA | | 6 |
| CK-19 | F: ATGGCCGAGCAGAACCGGAA | 308 bp | 7 |
| | R: CCATGAGCCGCTGGTACTCC | | 8 |
| Vim | F: CACCTACAGCCTCTACG | 170 bp | 9 |
| | R: AGCGGTCATTCAGCTC | | 10 |
| α-SMA | F: AGTACCCGATAGAACATGG | 153 bp | 11 |
| | R: TTTTCTCCCGGTTGGC | | 12 |
| CYP1B1 | F: CACCAAGGCTGAGACAGTGA | 230 bp | 13 |
| | R: GCCAGGTAAACTCCAAGCAC | | 14 |
| CYP2C9 | F: GGACAGAGACGACAAGCACA | 200 bp | 15 |
| | R: TGGTGGGGAGAAGGTCAAT | | 16 |
| CYP3A4 | F: TGTGCCTGAGAACACCAGAG | 201 bp | 17 |
| | R: GCAGAGGAGCCAAATCTACC | | 18 |
| CYP2E1 | F: CCGCAAGCATTTTGACTACA | 202 bp | 19 |
| | R: GCTCCTTCACCCTTTCAGAC | | 20 |
| CYP1A1 | F: AGGCTTTTACATCCCCAAGG | 197 bp | 21 |
| | R: GCAATGGTCTCACCGATACA | | 22 |
| β-Actin | F: TCACCACCACGGCCGAGCG | 350 bp | 23 |
| | R: TCTCCTTCTGCATCCTGTCG | | 24 |

Abbreviations: F, Forward; R, Reverse; bp, base pair; Alb, albumin; AFP, α-fetoprotein; CK-19, cytokeratin 19; Vim, vimentin; α-SMA, alpha-smooth muscle actin, CYP, cytochrome P450

### Results

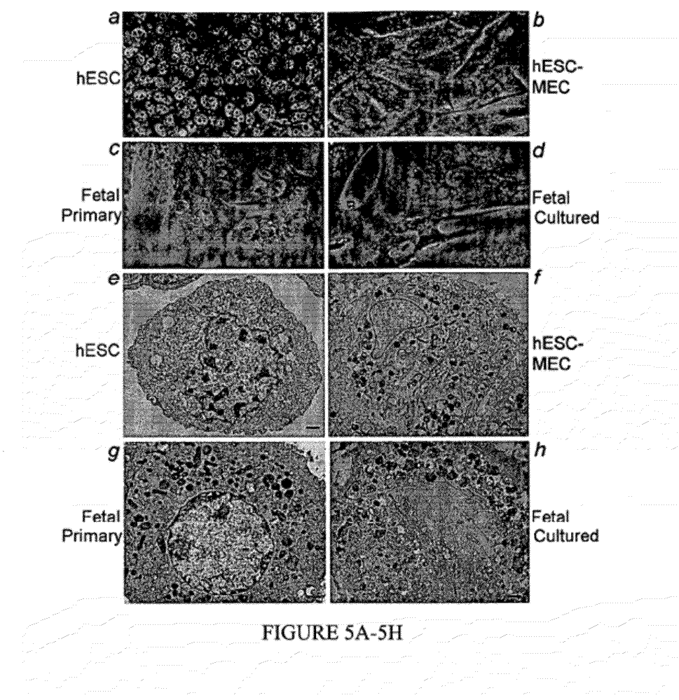

Characterization of hESC-derived cells showed fetal liver cell-like properties. Human ESC-derived putative mesenchymal stem cells were characterized for epithelial properties. In hESC-derived cells, properties associated with cells originating from either mesoderm or endoderm was simultaneously observed. In view of this conjoint meso-endoderm phenotype, these cells were designated hESC-derived Meso-Endoderm Cells (hESC-MEC). Human ESC-MEC resembled epithelial human fetal liver stem/progenitor cells that had been cultured for three or more passages (FH-Ep-P3). However, ultrastructural analysis with transmission electron microscopy revealed that morphology of hESC-MEC and FH-Ep-P3 differed from freshly isolated epithelial fetal liver stem/progenitor cells (FH-Ep-PP), and from undifferentiated WA01 hESC (FIG. 5). hESC-MEC and cultured fetal liver cells showed intermediate filaments along with complex cytoplasm, including multiple mitochondria, vacuoles and primary lysosomes, which was consistent with mesenchymal plus epithelial properties. To verify changes in cells during this process of spontaneous differentiation, immunostaining methods were used to examine expression of pluripotency-associated genes, e.g., OCT4, SSEA4 and TRA-1-60. These genes were expressed at lower levels in hESC-MEC, which was similar to FH-Ep-P3 cells, and different from undifferentiated hESC. Molecular assays with real-time polymerase chain reaction (RT-PCR) showed that hESC-MEC expressed mesenchymal genes—vimentin and α-SMA, along with epithelial genes—Alb, CK-19 and multiple Cyp450 genes, which reiterated similarities with conjoint mesenchymal and epithelial properties in FH-Ep-P3 cells (Inada et al., 2008b).

These results were verified by cytochemical stainings in that hESC-MEC contained markers of hepatocytes or biliary cells, including glycogen, G-6-P, GGT, as well as the mesenchymal marker, vimentin. Since gene promoters are well-known to be regulated by cellular cofactors in cell type-specific fashion, the activity of hepatic promoter constructs was examined after introducing these in hESC-MEC by lentiviral vectors (LV). Cells expressed Alb, transthyretrin (TTR) and α-1-antitrypsin (AAT) promoters, Alb>TTR>AAT, which was in accordance with correct intracellular contexts and permissiveness for transcription of hepatic genes (Inada et al., 2008b).

Studies of genome-wide gene expression with Affymetrix U133 Plus 2.0 Arrays in undifferentiated hESC, hESC-MEC and FH-Ep-P3 cells showed differential regulation of genes in multiple genetically-defined processes, functions and pathways. From 47,700 transcripts, 3780 genes (8%) were upregulated and 4134 genes (9%) were downregulated in hESC-MEC versus undifferentiated hESC. This resembled upregulation of 4688 genes (10%) and downregulation of 4951 genes (10%) in cultured FH-Ep-P3 fetal liver cells versus hESC (p=n.s.). In contrast, comparison of hESC-MEC and FH-Ep-P3 cells showed fewer genes were upregulated, 2115 genes (4%), or downregulated, 2332 genes (5%) (p<0.05). Therefore, although hESC-MEC and FH-Ep-P3 cells were not identical, both cell types showed greater divergence from undifferentiated hESC than from one another. This genetic convergence was substantiated by observing similarities in hESC-MEC and FH-Ep-P3 in cytokine-signaling networks directing cell differentiation, e.g., transforming growth factor (TGF)-β or bone morphogenetic protein (BMP) pathways, which control mesenchymal differentiation. Similarly, broad convergences in hESC-MEC and FH-Ep-P3 were found with array-based analysis of >500 cellular microRNAs, which may be involved in stem cell differentiation (not shown).

Multilineage differentiation of hESC-MEC was similar to fetal hepatoblasts. To verify the capacity of hESC-MEC in generating mesenchymal lineages, osteogenic, adipogenic and endothelial differentiation were induced over 3 weeks with established protocols in vitro (Dan et al., 2006; Inada et al., 2008b; Ria et al., 2008). This generated osteocytes, adipocytes and endothelial-like cells, which was similar to the generation of these lineages by fetal liver stem/progenitor cells (Inada et al., 2008b). Next, it was determined whether hepatic differentiation in hESC-MEC could be advanced by combinations of cytokines thought to be potent inducers of endoderm differentiation in stem cells, e.g., activin A, aFGF, hepatocyte growth factor, oncostatin M, Wnt and Notch antagonists, e.g., DKK-1 or γ-secretase inhibitor X, or the histone deacetylase inhibitor, trichostatin A. After culturing cells for 3 weeks with these substances, examination was carried out of appearance of or changes in hepatobiliary markers, e.g., glycogen, DPPIV, GGT, Alb, α-fetoprotein, hepatic nuclear factors, and other genes. However, these cytokines and permutations in cytokine combinations did not enhance hepatic differentiation in hESC-MEC. It should be noteworthy that differentiation protocols incorporating activin A induce endoderm differentiation in hESC (Phillips et al., 2007), whereas lack of their efficacy in hESC-MEC and FH-Ep-P3 cells suggested that alternative differentiation mechanisms applied. Nonetheless, taking the evidence together, it was concluded that naturally encountered meso-endoderm stage of fetal liver cells was also required during hepatic differentiation in stem cells.

Therapeutic functions of hESC-derived fetal liver-like cells. Although hESC-MEC exhibited an immature hepatic phenotype, presence of glycogen, G-6-P, Cyp450 and other relevant hepatic functions made these cells of interest for life-support in ALF. This possibility was examined in NOD/SCID mice with ALF induced by Rif and Phen over 3 d, followed on d 4 by the hepatotoxic pyrrolizidine alkaloid, MCT. This produced 50-70% liver necrosis, along with abnormal liver tests, coagulopathy, encephalopathy, and 90-100% mortality over several days. Mice with ALF were rescued after mature rat hepatocytes anchored to extracellular matrix-coated microcarriers had been transplanted in the peritoneal cavity. Conglomerates of transplanted cells and microcarriers are revascularized in the peritoneal cavity leading to preservation of metabolic functions in transplanted cells, secretion of secreted proteins in blood, and survival of transplanted cells for several weeks. It should be noteworthy that reseeding of the damaged liver with healthy hepatocytes was not required for liver regeneration in this model of ALF. Also, cells transplanted in the peritoneal cavity do not migrate to other organs, including the liver. Therefore, cell transplantation in the peritoneal cavity was used to demonstrate the hepatic support capacity of transplanted hESC-MEC (Demetriou et al., 1986; Gupta et al., 1994).

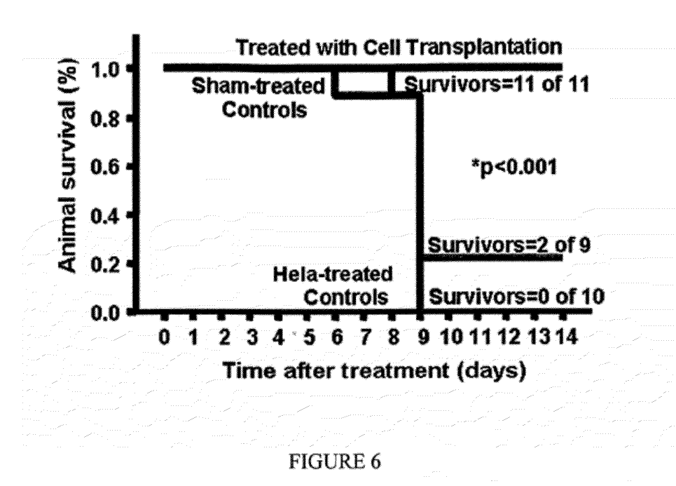

After inducing ALF with Rif, Phen and 125 mg/kg MCT, mice were divided into groups with transplantation of 4-6×10⁶ hESC-MEC (n=11); 5×10⁶ HeLa human cervical cells (irrelevant control) (n=10); and vehicle alone (sham treatment) (n=9). After 2 weeks, all 11 hESC-MEC-treated mice survived (100%), only 2 sham-treated mice survived (22%), and no HeLa-treated mice survived (0%) (p<0.001, ANOVA) (FIG. 6). Hypoglycemia was excluded as a cause of death by measuring blood glucose in several animals. Mice treated with hESC-MEC remained healthy without onset of encephalopathy, whereas vehicle- and HeLa-treated mice developed grade III-IV encephalopathy (p<0.05). Human Sex-determining Region Y (SRY) was demonstrated in transplanted hESC-MEC by DNA PCR (Wang et al., 2002). Moreover, in 2 of 11 mice with ALF, 2 weeks after transplanting hESC-MEC, 0.48 and 0.21 ng/ml human albumin were detected in blood (normal, 40 μg/ml). Human albumin was absent in vehicle- or HeLa-treated mice. Production and secretion of albumin in small amounts by transplanted hESC-MEC was in agreement with incomplete hepatic differentiation and immature phenotype.

To verify that hESC-MEC could rescue mice with more severe ALF, cell transplantation studies were repeated by giving more MCT (160 mg/kg) to Rif and Phen-treated mice. In this situation, 38% of hESC-MEC-treated mice survived compared with survival of only 13% after sham treatment over 2 weeks (n=8 each) (p<0.05).

The liver of sham-treated mice was grossly abnormal with edema and hemorrhagic necrosis, while the liver of mice treated with cells was healthy. Transplantation of hESC-MEC substantially restored liver histology to normal and prevalence of Ki67-expressing hepatocytes increased, indicating superior liver regeneration.

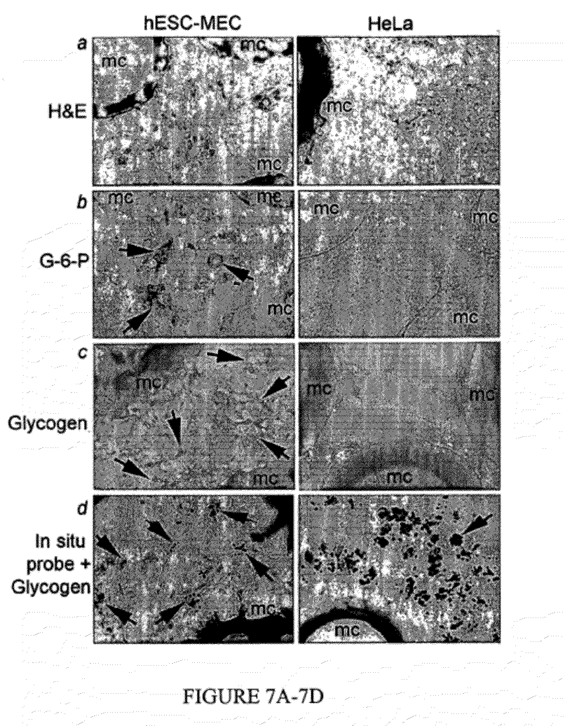

Transplanted human cells were identified in situ with a pancentromeric human probe (Benten et al., 2006). Transplanted hESC-MEC contained glycogen and G-6-P, whereas these hepatic markers were absent in HeLa cells, as expected (FIG. 7A-7D). Also, transplantation of hESC-MEC after transduction with Alb-GFP LV demonstrated hESC-MEC expressed hepatic function. Similarly, transplantation of hESC-MEC in the liver of NOD/SCID mice indicated that cells engrafted in the liver parenchyma and contained glycogen, which was in further agreement with hepatic function.

To determine whether mice with ALF were rescued by hepatic support from transplanted cells or paracrine effects from proteins secreted by transplanted cells, conditioned medium from hESC-MEC were injected into mice. This failed to improve survival and mice died over 7 d, indicating intact hESC-MEC are necessary for improving mortality. Multiple cytokines were identified in the conditioned medium using a cytokine array.

Transcriptional differences in pathways of hepatic stress and toxicity were analyzed 3 and 7 d after ALF with an array of 84 genes in sham- and hESC-MEC-treated mice (n=3 each). This showed significant oxidative stress and hepatic toxicity in sham-treated mice with perturbations in Cyp450 and other metabolic genes, chemokines, and cell cycle checkpoint controls, e.g., p21, these changes were attenuated in hESC-MEC-treated mice (Table 4), consistent with hepatic recovery.

Transplantation of hESC-MEC in NOD/SCID mice did not produce neoplasia. In formal assays of tumorigenicity, hESC-MEC were injected subcutaneously, and no tumors were observed over a period of at least 3 months.

Additional transplantation studies investigated whether adult human hepatocytes, fetal human liver cells, and hTERT-FH-B cells could rescue NOD/SCID mice with ALF induced by Rif, Phen and MCT. Transplantation of 5×10⁶ cells with Cytodex3 microcarriers in the peritoneal cavity produced 90%, 55% and 40% survival in recipients of adult hepatocytes, fetal liver cells, or hTERT-FHB, respectively, versus 0% survival in sham-treated mice with injection of microcarriers alone, p<0.05. Presence of transplanted cells was verified by in situ hybridization with human-specific pancentromeric probe. Transplanted cells expressed albumin mRNA, and serum human albumin was also detected in recipients. Neither aberrant proliferation of cells nor any tumorigenicity was observed after cell transplantation in mice.

TABLE 4
Livers from sham-treated versus normal mice and
hESC-MEC cell transplantation versus normal mice.

| | | S | p | S | p | Cells | p | Cells | p |
|---|---|---|---|---|---|---|---|---|---|
| Gene Name | Symbol | D3/N | value | D7/N | value | D3/N | value | D7/N | value |
| Bcl2-associated | Bax | 1.5 | 0.0223 | 2.3 | 0.0048 | 2.0 | 0.0014 | 1.9 | 0.0020 |
| X protein | | | | | | | | | |
| Chemokine (C-C | Ccl3 | 5.4 | 0.0084 | 30.7 | 0.0030 | 6.3 | 0.0170 | 3.4 | 0.0165 |
| motif) ligand 3 | | | | | | | | | |
| Chemokine (C-C | Ccl4 | 3.4 | 0.0077 | 21.5 | 0.0031 | 5.4 | 0.0081 | 2.2 | 0.0338 |
| motif) ligand 4 | | | | | | | | | |
| Cyclin-dependent | Cdkn- | 105.0 | 0.0005 | 92.0 | 0.0006 | 45.2 | 0.0009 | 40.6 | 0.0009 |
| kinase inhibitor | 1a | | | | | | | | |
| 1A (P21) | | | | | | | | | |
| Cytochrome | Cyp2- | −46.3 | 0.0001 | −106.9 | 0.0017 | −6.8 | 0.0134 | −3.6 | 0.0217 |
| P450, family 2, | c29 | | | | | | | | |
| subfamily c, | | | | | | | | | |
| Polypeptide 29 | | | | | | | | | |
| Cytochrome | Cyp3- | −13.8 | 0.0001 | −27.7 | 0.0002 | −21.5 | 0.0160 | −6.3 | 0.0003 |
| P450, family 3, | a11 | | | | | | | | |
| subfamily a, | | | | | | | | | |
| Polypeptide 11 | | | | | | | | | |
| Epoxide | Ephx2 | −4.2 | 0.0246 | −10.4 | 0.0037 | −3.6 | 0.0165 | −2.9 | 0.0141 |
| hydrolase 2, | | | | | | | | | |
| cytoplasmic | | | | | | | | | |
| Metallothionein 2 | Mt2 | 5.7 | 0.0009 | 7.8 | 0.0364 | 4.9 | 0.0032 | 4.0 | 0.0070 |
| Superoxide | Sod1 | −2.7 | 0.0002 | −3.6 | 0.0001 | −1.9 | 0.0154 | −1.7 | 0.0076 |
| dismutase 1, | | | | | | | | | |
| soluble | | | | | | | | | |

S, Sham; D, Day after induction of acute liver failure; Cells, hESC-MEC; N, normal liver; − values indicate downregulation in folds above normal control liver. Data are from three replicate animals in each condition.

### Discussion

These findings established that conjoint meso-endoderm stage of fetal hepatic endoderm development was equally critical in the hepatic differentiation of hESC-derived cells. Although this represented an early stage of hepatic lineage development, hESC-derived cells with meso-endoderm phenotype were able to support the failing liver and thereby facilitated recovery of damaged liver cells.

These studies of stem cell differentiation were in agreement with the putative origin of mesoderm and endoderm from shared mesendoderm precursor cells (Rodaway et al., 2001). Appearance of conjoint epithelial and mesenchymal properties in hESC-MEC indicated that these cells shared their identity with naturally occurring fetal human liver stem/progenitor cells (Inada et al., 2008a,b). The meso-endoderm state of hESC-MEC is thus considered to represent an early state of naturally occurring fetal liver stem/progenitor cells, resembling mesendodermal phenotype of cells during embryonic gastrulation. Expression at lower levels in hESC-MEC, as well as in fetal liver stem/progenitor cells, of Oct4, SSEA4 and TRA-1-60 transcription factors, which reflect pluripotency, was in agreement with cell differentiation and lineage progression.

Despite being immature, hESC-derived early fetal liver-like cells expressed critical functions, such as glycogen storage and glucose metabolism, which are required for liver support, as demonstrated in the present studies of cell therapy in ALF. These studies established mechanisms in how hESC-derived early fetal liver-like cells could rescue animals with toxic drug-induced ALF. As drug toxicity is a major cause of ALF in United States, Europe and elsewhere, this should be highly significant. hESC-derived cells engrafted, survived and functioned in animals, and promoted regeneration of the native liver through extrahepatic liver support.

Previously, uncertainties had emerged in mechanisms of liver regeneration following hepatocyte transplantation in ALF, because transplantation of intact cells, cell fragments or conditioned medium from cells was equally effective in animals (Baumgartner et al., 1983; Grundmann et al., 1986; Makowka et al., 1980). By contrast, in the present studies, intact hESC-derived cells were required for rescuing animals with ALF. Although hESC-MEC secreted numerous cytokines, treatment of animals with conditioned medium containing these cytokines alone did not rescue animals with ALF. This difference from previous studies may be a consequence of more human-like liver injury in the present NOD/SCID mouse model. The role of ataxia telangiectasia mutant gene pathway in oxidative stress, DNA damage and p21-dependent checkpoint controls in this model offers new ways to approach drug development or other therapeutic interventions. Moreover, the ability of residual hepatocytes to repair themselves and regenerate the liver resembled the clinical situation in people (Quaglia et al., 2008), which will further enhance the value of this animal model of ALF and of the therapeutic principles gained by studies within the context of pathophysiological mechanisms in ALF.

This therapeutic potential of hESC-derived early fetal liver-like cells, as well as of adult hepatocytes, primary fetal liver stem/progenitor cells, and immortalized fetal stem/progenitor cells in ALF should be highly attractive for clinical applications of cell therapy. Recovery through extrahepatic cell transplantation in the peritoneal cavity will be far simpler than reseeding of the liver with cells, which requires hazardous invasive means for cell transplantation, such as intravascular injection of cells in the setting of coagulopathies. The need for only short-term liver support in ALF should permit applications of pre-prepared and frozen cells derived from allogeneic stem cells, which should be helpful for clinical applications. Similarly, provision of liver support in the setting of incipient liver failure, e.g., after liver surgery or organ failure due to other causes, as well as in people with chronic liver insufficiency, will constitute suitable applications of the proposed methods and principles.

All publications mentioned herein are hereby incorporated in their entirety into the subject application. While the foregoing invention has been described in some detail for purposes of clarity and understanding, it will be appreciated by one skilled in the art, from a reading of the disclosure, that various changes in form and detail can be made without departing from the true scope of the invention in the appended claims.

### REFERENCES

Baumgartner D, LaPlante-O'Neill P M, Sutherland D E, Najarian J S (1983) Effects of intrasplenic injection of hepatocytes, hepatocyte fragments and hepatocyte culture supernatants on D-galactosamine-induced liver failure in rats. Eur Surg Res 15: 129-135.
Benten D, Cheng K, Gupta S (2006) Identification of transplanted human cells in animal tissues. Methods Mol Biol 326: 189-201.
Bhuller, Y., Jeng, W., and Wells, P. G. 2006. Variable In Vivo Embryoprotective Role for Ataxia-Telangiectasia-Mutated against Constitutive and Phenyloin-Enhanced Oxidative Stress in Atm Knockout Mice. Toxicol Sci 93: 146-55.
Braun, K. M., Degen, J. L., and Sandgren, E. P. 2000. Hepatocyte transplantation in a model of toxin-induced liver disease: variable therapeutic effect during replacement of damaged parenchyma by donor cells. Nat Med 6: 320-6.
Cho, J., Joseph, B., Sappal, B. S., Giri, R. K., Wang, R., Ludlow, J., Susick, R., and Gupta, S. 2004. Analysis of the functional integrity of cryopreserved human liver cells including xenografting in immunodeficient mice to address suitability for clinical applications. Liver International 24: 361-70.
Chowdhury, A., Santra, A., Bhattacharjee, K., Ghatak, S., Saha, D. R., and Dhali, G. K. 2006. Mitochondrial oxidative stress and permeability transition in isoniazid and rifampicin induced liver injury in mice. J Hepatol 45: 117-26.
Dan Y Y, Riehle K J, Lazaro C, Teoh N, Hague J, et al. (2006) Isolation of multipotent progenitor cells from human fetal liver capable of differentiating into liver and mesenchymal lineages. Proc Natl Acad Sci USA 103: 9912-9917.
Demetriou, A. A., Whiting, J. F., Feldman, D., Levenson, S. M., Chowdhury, N. R., Moscioni, A. D., Kram, M., and Chowdhury, J R. 1986. Replacement of liver function in rats by transplantation of microcarrier-attached hepatocytes. Science 233: 1190-92.
Fisher, R. A. and Strom, S. C. 2006. Human hepatocyte transplantation: worldwide results. Transplantation 82: 441-9.
Gagandeep, S., Sokhi, R., Slehria, S., Gorla, G. R., Furgiuele, J., DePinho, R. A., and Gupta, S. 2000. Hepatocyte transplantation improves survival in mice with liver toxicity induced by hepatic overexpression of Mad1 transcription factor. Mol Ther 1: 358-365.
Gorla, G. R., Malhi, H., and Gupta, S. 2001. Polyploidy associated with oxidative DNA injury attenuates proliferative potential of cells. J Cell Sci 114: 2943-2951.
Grundmann R, Koebe H G, Waters W (1986) Transplantation of cryopreserved hepatocytes or liver cytosol injection in the treatment of acute liver failure in rats. Res Exp Med (Berl) 186: 141-149.
Gupta, S., Rajvanshi, P., Irani, A. N., Palestro, C. J., and Bhargava, K. K. 2000. Integration and proliferation of transplanted cells in hepatic parenchyma following D-galactosamine-induced acute injury in F344 rats. J Pathol 190: 203-210.
Gupta, S., Rajvanshi, P., Malhi, H., Sokhi, R. P., Slehria, S., Vasa, S. R. G., Dabeva, M., Shafritz, D. A., and Kerr, A. 2000. Cell transplantation causes loss of gap junctions and activates GGT expression permanently in host liver. Am J Physiol Gastroint Liver Physiol 279: G815-G826.
Gupta, S., Vemuru, R. P., Lee, C-D., Yerneni, P., Aragona, E., and Burk, R. D. 1994. Hepatocytes exhibit superior transgene expression after transplantation into liver and spleen compared with peritoneal cavity or dorsal fat pad: Implications for hepatic gene therapy. Human Gene Ther 5: 959-967.
Habibullah, C. M., Syed, I. H., Qamar, A., and Taher-Uz, Z. 1994. Human fetal hepatocyte transplantation in patients with fulminant hepatic failure. Transplantation 58: 951-2.
Inada M, Benten D, Cheng K, Joseph B, Berishvili E, Badve S, Logdberg L, Dabeva M, Gupta S. (2008a) Stage-specific regulation of adhesion molecule expression segregates epithelial stem/progenitor cells in fetal and adult human livers. Hepatology International 2: 50-62.
Inada M, Follenzi A, Cheng K, Surana M, Joseph B, et al. (2008b) Phenotype reversion in fetal human liver epithelial cells identifies the role of an intermediate meso-endodermal stage before hepatic maturation. J Cell Sci 121: 1002-1013.
Joseph, B., Malhi, H., Bhargava, K. K., Palestro, C. J., McCuskey, R. S., and Gupta, S. 2002. Kupffer cells participate in early clearance of syngeneic hepatocytes transplanted in the rat liver. Gastroenterology 123: 1677-1685.
Kanu, N. and Behrens, A. 2008. ATMINistrating ATM signalling: regulation of ATM by ATMIN. Cell Cycle 7: 3483-6.
Krohn, N., Kapoor, S., Enami, Y., Follenzi, A., Bandi, S., Joseph, B., and Gupta, S. 2009. Hepatocyte transplantation-induced liver inflammation is driven by cytokines-chemokines associated with neutrophils and Kupffer cells. Gastroenterology 136(5):1806-17.
Kumaran, V., Benten, D., Follenzi, A., Joseph, B., Sarkar, R., and Gupta, S. 2005. Transplantation of endothelial cells corrects the phenotype in hemophilia A mice. J Thromb Haemost 3: 2022-31.
Kuribayashi, K. and El-Deiry, W. S. 2008. Regulation of programmed cell death by the p53 pathway. Adv Exp Med Biol 615: 201-21.
Lu, S., Shen, K. C., Wang, Y., Brooks, S. C., and Wang, Y. A. 2005. Impaired hepatocyte survival and liver regeneration in Atm-deficient mice. Hum Mol Genet. 14: 3019-25.
Makowka L, Falk R E, Rotstein L E, Falk J A, Nossal N, et al. (1980) Cellular transplantation in the treatment of experimental hepatic failure. Science 210: 901-903.
Murray, K. F., Hadzic, N., Wirth, S., Bassett, M., and Kelly, D. 2008. Drug-related hepatotoxicity and acute liver failure. J Pediatr Gastroenterol Nutr 47: 395-405.
Nakagawa M, Koyanagi M, Tanabe K, Takahashi K, Ichisaka T, et al. (2008) Generation of induced pluripotent stem cells without Myc from mouse and human fibroblasts. Nat Biotechnol 26: 101-106.
Olivier E N, Rybicki A C, Bouhassira E E (2006) Differentiation of human embryonic stem cells into bipotent mesenchymal stem cells. Stem Cells 24: 1914-1922.
Phillips B W, Hentze H, Rust W L, Chen Q P, Chipperfield H, et al. (2007) Directed differentiation of human embryonic stem cells into the pancreatic endocrine lineage. Stem Cells Dev 16: 561-578.
Quaglia, A. 2008. Auxiliary transplantation for acute liver failure: Histopathological study of native liver regeneration. Liver Transpl 14: 1437-48.
Quaglia A, Portmann B C, Knisely A S, Srinivasan P, Muiesan P, et al. (2008) Auxiliary transplantation for acute liver failure: Histopathological study of native liver regeneration. Liver Transpl 14: 1437-1448.
Rajvanshi, P., Kerr, A., Bhargava, K. K., Burk, R. D., and Gupta, S. 1996. Studies of liver repopulation using the dipeptidyl peptidase IV deficient rat and other rodent recipients: cell size and structure relationships regulate capacity for increased transplanted hepatocyte mass in the liver lobule. Hepatology 23: 482-496.
Ria R, Piccoli C, Cirulli T, Falzetti F, Mangialardi G, et al. (2008) Endothelial differentiation of hematopoietic stem and progenitor cells from patients with multiple myeloma. Clin Cancer Res 14: 1678-1685.
Rodaway A, Patient R (2001) Mesendoderm. an ancient germ layer? Cell 105: 169-172.
Sigal, S. H., Rajvanshi, P., Gorla, G. R., Saxena, R., Sokhi, R. P., Gebhardt, D. F., Jr., Reid, L. M., and Gupta, S. 1996. Partial hepatectomy-induced polyploidy attenuates hepatocyte replication and activates cell aging events. Am J Physiol 276: G1260-G1272.
Stickel F, Egerer G, Seitz H K. 2000. Hepatotoxicity of botanicals. Public Health Nutr 3:113-24.
Thomson J A, Itskovitz-Eldor J, Shapiro S S, Waknitz M A, Swiergiel J J, et al. (1998) Embryonic stem cell lines derived from human blastocysts. Science 282: 1145-1147.
Wang L J, Chen Y M, George D, Smets F, Sokal E M, et al. (2002) Engraftment assessment in human and mouse liver tissue after sex-mismatched liver cell transplantation by real-time quantitative PCR for Y chromosome sequences. Liver Transpl 8: 822-828.
Wang, Y. P., Yan, J., Beger, R. D., Fu, P. P., and Chou, M. W. 2005. Metabolic activation of the tumorigenic pyrrolizidine alkaloid, monocrotaline, leading to DNA adduct formation in vivo. Cancer Lett 226: 27-35.
Wege H, Le H T, Chui M S, Liu L, Wu J, Giri R K, Malhi H, Sappal B S, Kumaran V, Gupta S, Zern M A. (2003) Telomerase reconstitution immortalizes human fetal hepatocytes without disrupting their differentiation potential. Gastroenterology 124: 432-444.
Wu Y M, Joseph B, Berishvili E, Kumaran V, Gupta S. 2008. Hepatocyte transplantation and drug-induced perturbations in liver cell compartments. Hepatology 47: 279-87.
Zalzman M, Gupta S, Giri R K, Berkovich I, Sappal B S, Karnieli O, Zern M A, Fleischer N, Efrat S. (2003) Reversal of hyperglycemia in mice using human expandable insulin-producing cells differentiated from fetal liver progenitor cells. Proc Natl Acad Sci USA 100: 7253-8.
Zhang, J. and Chen, X. 2008. Posttranscriptional regulation of p53 and its targets by RNA-binding proteins. Curr Mol Med 8: 845-9.

1. A method of treating liver failure in a subject comprising transplanting hepatocytes or stem cells or progenitor cells in an extrahepatic site in the subject in an amount sufficient to induce liver regeneration, wherein the transplanted hepatocytes or stem cells or progenitor cells are attached to extracellular matrix-coated microcarriers.

2. The method of claim 1, wherein the hepatocytes or stem cells or progenitor cells are transplanted into the peritoneal cavity.

3. The method of claim 1, wherein mature hepatocytes are transplanted into the subject.

4. The method of claim 1, wherein the transplanted cells are fetal liver stem or progenitor cells, or cells derived from embryonic or equivalent stem or progenitor cells.

5. The method of claim 1, wherein the transplanted cells are mesenchymal stem cells or stem cells with mesenchymal and epithelial phenotype.

6. The method of claim 5, wherein the mesenchymal cells have a meso-endoderm phenotype.

7. The method of claim 5, wherein the mesenchymal or meso-endoderm stem cells are obtained by differentiation of cultured human embryonic stem cells or cells with equivalent stem cell potential.

8. The method of claim 1, wherein the transplanted cells are stem cell-derived liver-like cells.

9. The method of claim 8, wherein the transplated cells are derived from human embryonic stem cells (hESC) or induced pluripotent stem (iPS) cells.

10. The method of claim 1, wherein the microcarriers are collagen-coated.

11. The method of claim 1, wherein the extracellular matrix-coated microcarriers are biodegradable.

12. The method of claim 1, wherein the microcarriers are spherical in shape.

13. The method of claim 1, wherein the microcarriers have dimensions of 100-300 μm.

14. The method of claim 1, wherein the subject has acute liver failure, incipient liver failure or ongoing liver failure.
